(19) United States
(12) Patent Application Publication
(10) Pub. No.: US 2004/0255957 A1
(43) Pub. Date: Dec. 23, 2004

(54) Method and system for treating vulnerable plaque

(76) Inventor: Robert Cafferata, Santa Rosa, CA (US)

Correspondence Address: FRANK C. NICHOLAS, CARDINAL LAW GROUP, Suite 2000, 1603 Orrington Avenue, Evanston, IL, 60201, US

(21) Appl. No.: 10/625,809

(22) Filed: Jul. 23, 2003

Related U.S. Application Data
(63) Continuation-in-part of application No. 10/427,680, filed on May 1, 2003.

(51) Int. Cl. A61B 19/00; A61F 2/00; A61B 18/04

(52) U.S. Cl. 128/898; 606/32; 607/100

(57) ABSTRACT

A method and system for treating a vulnerable plaque associated with a blood vessel of a patient is disclosed. The method includes rupturing a fibrous cap of the vulnerable plaque. A portion of liquid contents of the vulnerable plaque are released into a blood vessel lumen as a result of the fibrous cap rupture. The method further includes capturing at least one of any emboli present within the blood vessel as a result of the fibrous cap rupture. The system includes a rupture device that ruptures a fibrous cap of the vulnerable plaque and a capture device that captures at lest one embolus within the blood vessel.

### PRIORITY

This application claims priority as a continuation in part to U.S. patent application Ser. No. 10/127,680 filed May 1, 2003.

### TECHNICAL FIELD OF THE INVENTION

The present invention relates generally to the field of vascular therapies. More particularly, the invention relates to a method and system for treating a vulnerable plaque associated with a blood vessel of a patient.

### BACKGROUND OF THE INVENTION

Heart disease, specifically coronary artery disease, is a major cause of death, disability, and healthcare expense. Until recently, most heart disease was considered to be primarily the result of a progressive increase of hard plaque in the coronary arteries. This atherosclerotic disease process of hard plaques leads to a critical narrowing (stenosis) of the affected coronary artery and produces anginal syndromes, known commonly as chest pain. The progression of the narrowing reduces blood flow, triggering the formation of a blood clot. The clot may choke off the flow of oxygen rich blood (ischemia) to heart muscles, causing a heart attack. Alternatively, the clot may break off and lodge in another organ vessel such as the brain resulting in a thrombotic stroke.

Within the past decade, evidence has emerged expanding the paradigm of atherosclerosis, coronary artery disease, and heart attacks. While the build up of hard plaque may produce angina and severe ischemia in the coronary arteries, new clinical data now suggests that the rupture of sometimes non-occlusive, vulnerable plaques causes the vast majority of heart attacks. The rate is estimated as high as 60-80 percent. In many instances vulnerable plaques do not impinge on the vessel lumen, rather, much like an abscess they are ingrained under the arterial wall. For this reason, conventional angiography or fluoroscopy techniques are unlikely to detect the vulnerable plaque. Due to the difficulty associated with their detection and because angina is not typically produced, vulnerable plaques may be more dangerous than other plaques that cause pain.

The majority of vulnerable plaques include a lipid pool, necrotic smooth muscle (endothelial) cells, and a dense infiltrate of macrophages contained by a thin fibrous cap, some of which are only two micrometers thick or less. The lipid pool is believed to be formed as a result of pathological process involving low density lipoprotein (LDL), macrophages, and the inflammatory process. The macrophages oxidize the LDL producing foam cells. The macrophages, foam cells, and associated endothelial cells release various substances, such as tumor necrosis factor, tissue factor, and matrix proteinases. These substances can result in generalized cell necrosis and apoptosis, pro-coagulation, and weakening of the fibrous cap. The inflammation process may weaken the fibrous cap to the extent that sufficient mechanical stress, such as that produced by increased blood pressure, may result in rupture. The lipid core and other contents of the vulnerable plaque (emboli) may then spill into the blood stream thereby initiating a clotting cascade. The cascade produces a blood clot (thrombosis) that potentially results in a heart attack and/or stroke. The process is exacerbated due to the release of collagen and other plaque components (e.g., tissue factor), which enhance clotting upon their release.

Several strategies have been developed for the detection (e.g., diagnosis and localization) of vulnerable plaques. One strategy involves the measurement of temperature within a blood vessel. For example, vulnerable plaque tissue temperature is generally elevated compared to healthy vascular tissue. Measurement of this temperature discrepancy may allow detection of the vulnerable plaque.

Another detection strategy involves labeling vulnerable plaque with a marker. The marker substance may be specific for a component and/or characteristic of the vulnerable plaque. For example, the marker may have an affinity for the vulnerable plaque, more so than for healthy tissue. Detection of the marker may thus allow detection of the vulnerable plaque. Alternatively, the marker may not necessarily have an affinity for the vulnerable plaque, but will simply change properties while associated with the vulnerable plaque. The property change may be detected and thus allow detection of the vulnerable plaque.

Regardless of the strategy used for detection, a formidable problem remains in the treatment of the vulnerable plaque. Without appropriate treatment, the vulnerable plaque may rupture and subsequently release embolic material and cause great risk to the patient, especially when the patient is not in a clinical setting. Drug and other therapies exist that may reduce the size and chance of vulnerable plaque rupture over a relatively long time frame. These therapies, however, may not be desirable or effective for all patients, including those having vulnerable plaques on the immediate verge of rupture. With such therapies, accidental or unanticipated rupture of these truly vulnerable plaques may occur in a non-clinical setting. Therefore, it would be desirable to provide a treatment strategy that would provide relatively immediate treatment of the vulnerable plaque within a clinical setting. Furthermore, it would be desirable for such a treatment strategy to prevent any embolic material from escaping and causing risk to the patient.

Accordingly, it would be desirable to provide a strategy for treating vulnerable plaque that would overcome the aforementioned and other disadvantages.

### SUMMARY OF THE INVENTION

One aspect of the invention provides a method of treating a vulnerable plaque associated with a blood vessel of a patient. The method includes rupturing the fibrous cap of the vulnerable plaque. A portion of liquid contents of the vulnerable plaque are released into a blood vessel lumen as a result of the fibrous cap rupture. The method further includes capturing at least one of any emboli present within the blood vessel as a result of the fibrous cap rupture.

Another aspect of the invention provides a system for treating a vulnerable plaque associated with a blood vessel of a patient. The system includes a rupture device that ruptures a fibrous cap of the vulnerable plaque and a capture device that captures at least one embolus within the blood vessel.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is schematic view of a system for treating a vulnerable plaque associated with a blood vessel of a patient, in accordance with one embodiment of the present invention;

FIG. 2 is a flow chart of a method of treating a vulnerable plaque associated with a blood vessel of a patient, in accordance with one embodiment of the present invention;

FIG. 3 is a schematic view of a patient undergoing the vulnerable plaque treatment method of FIG. 2;

FIG. 4 is a schematic view of a temperature sensing device for vulnerable plaque detection in a blood vessel, in accordance with one embodiment of the present invention;

FIG. 5 is a schematic view of an incising device for vulnerable plaque fibrous cap rupture in a blood vessel, in accordance with one embodiment of the present invention;

FIG. 6 is a schematic view of an ultrasonic device for vulnerable plaque fibrous cap rupture in a blood vessel, in accordance with one embodiment of the present invention;

FIGS. 7A, 7B, and 7C are schematic views sequentially depicting a combination device for treating a vulnerable plaque, in accordance with one embodiment of the present invention; and

FIG. 8 is schematic view of content release from a ruptured vulnerable plaque and capture of emboli within a blood vessel, in accordance with one embodiment of the present invention.

### DESCRIPTION OF THE PRESENTLY PREFERRED EMBODIMENTS

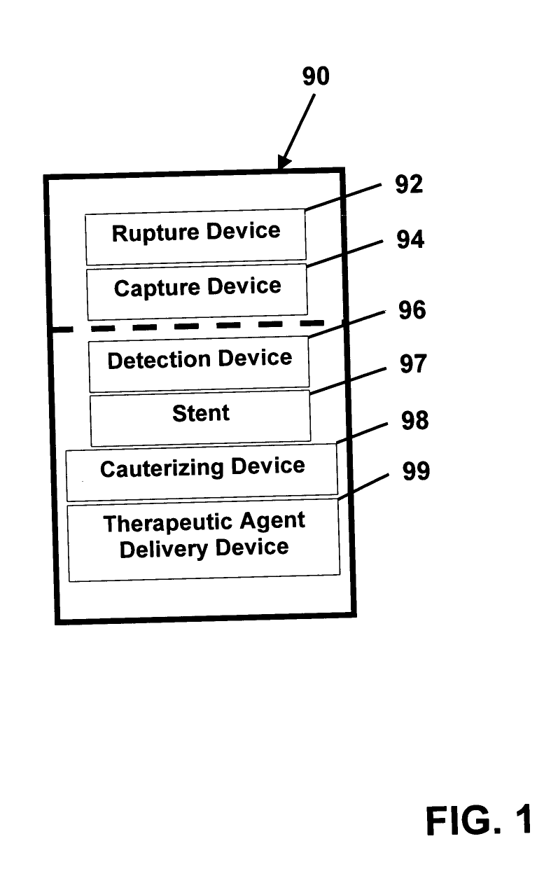

Referring to the drawings, wherein like reference numerals refer to like elements, a system 90 for treating a vulnerable plaque (VP) associated with a blood vessel of a patient is shown in FIG. 1. The system 90 includes a rupture device 92 that ruptures a fibrous cap of the vulnerable plaque and a capture device 94 that captures emboli within the blood vessel. The system 90 may optionally include a detection device 96 that detects the vulnerable plaque, a stent 97 operably attached to the rupture device 92, a cauterizing device 98 that cauterizes the vulnerable plaque, and/or a therapeutic agent delivery device 99. Specific examples of system 90 components as well as functional descriptions are provided in the following description of a method of treating a vulnerable plaque.

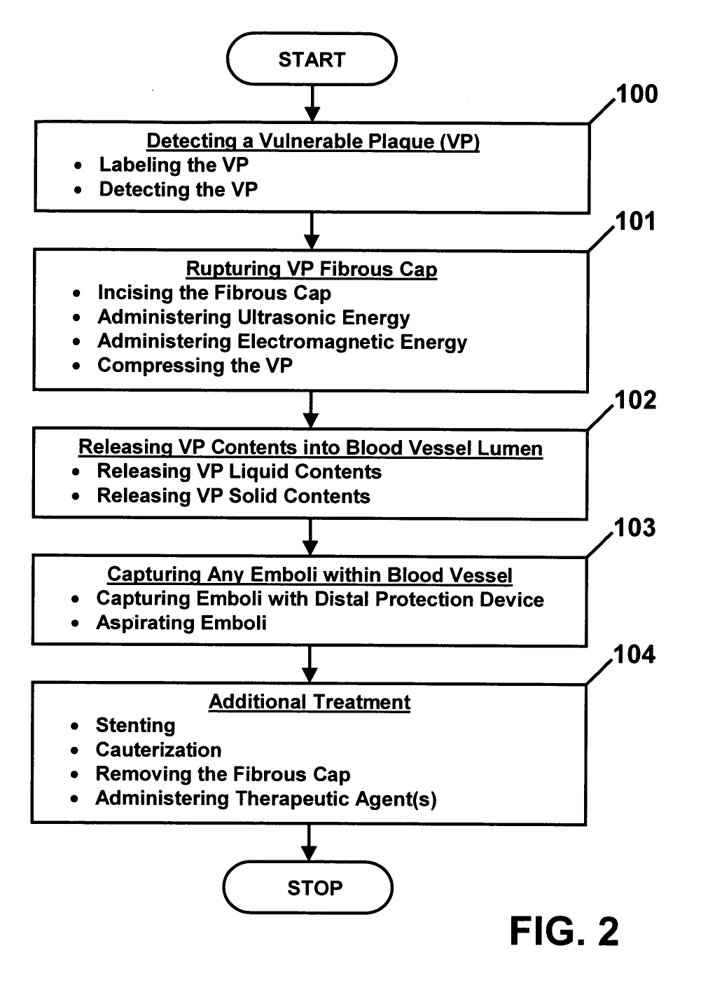

FIG. 2 is a flow chart of a method of treating a vulnerable plaque associated with a blood vessel of a patient, in accordance with one embodiment of the present invention. A vulnerable plaque is distinguishable from other types of plaque, including hard plaques, by the presence of a relatively thin fibrous cap. The vulnerable plaque fibrous cap retains a pool of lipids and other contents, which may be released into the blood vessel upon rupture of the cap. The released contents and any resulting blood clots constitute emboli that can lodge in a blood vessel thereby posing a risk to the patient. Vulnerable plaques, unlike hard plaques, are generally non-occlusive and, as such, may not produce angina. The following description pertains to treatment of these vulnerable plaques.

Those skilled in the art will recognize that although the present invention is described primarily in the context of treating a vulnerable plaque while using specific treatment devices, the inventor contemplates a broader method of application. Any number of treatment devices capable of performing the prescribed function(s) may be compatible with the present invention. Furthermore, the treatment of the vulnerable plaque is not limited to the described methodology. Numerous modifications, substitutions, and variations may be made to the method and system while providing effective vulnerable plaque treatment consistent with the present invention.

In the following description, vulnerable plaque treatment is described in the context of a catheterization detection and treatment procedure for a patient. The vulnerable plaque may be treated in a clinical setting thereby allowing for controlled treatment in an environment in which immediate care is given. Treating the vulnerable plaque in a manner according to the present invention may prevent the accidental or unanticipated release of emboli in a non-clinical setting. As such, complications stemming from vulnerable plaque rupture, such as heart attack and stroke, may be avoided. It should be noted that the terms “detect” and derivatives thereof, when used in regard to vulnerable plaque, refer to the diagnosis and localization of the lesion.

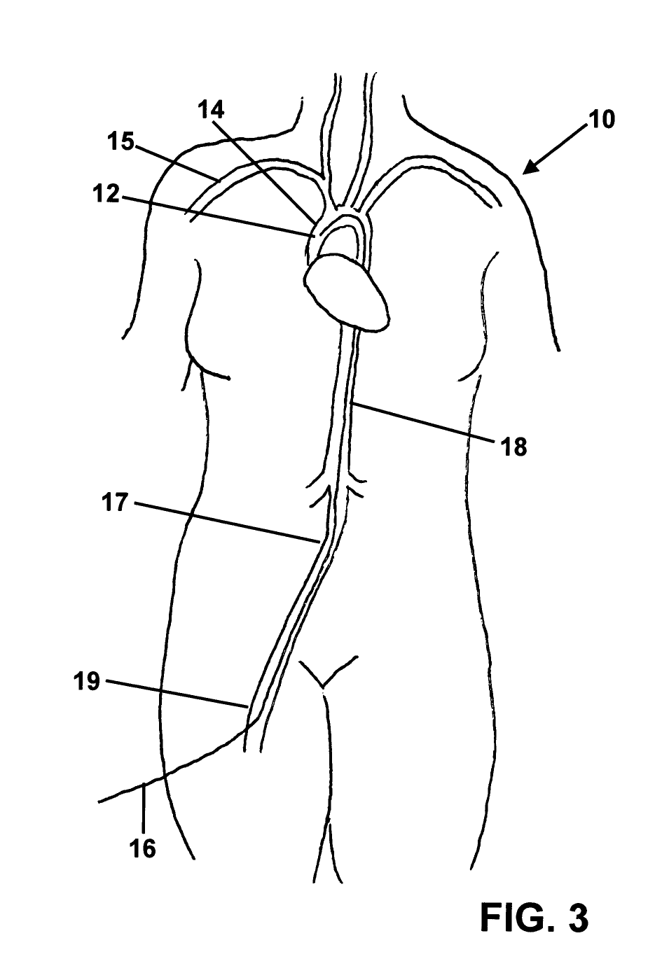

As shown in FIG. 3, patient 10 diagnostic site 12, which in this case is in an aortic vessel 14, may be accessed through various blood vessels. In one embodiment, the diagnostic site 12 may be accessed percutaneously through an incision made in patient 10 femoral artery 19. In another embodiment, another vessel such as a subclavian artery 15 may be used to access the diagnostic site 12. Devices 16 such as catheters and guidewires for detection and/or treatment of the vulnerable plaque may be advanced to the diagnostic site 12 through a vessel pathway, which in this case includes an iliac artery 17 and abdominal aorta 18. It is important to note that pathways and treatment site other than the ones described may be used with the present invention. In addition, the described order of events may be varied to achieve vulnerable plaque detection and treatment.

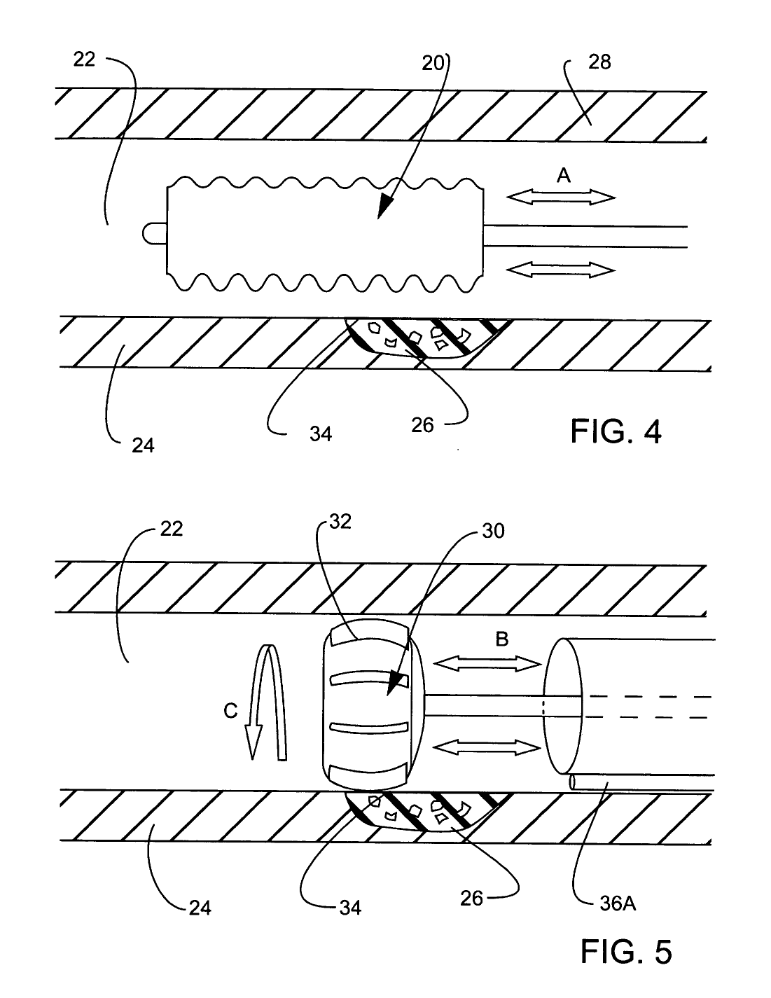

Referring again to FIG. 2, treatment of the vulnerable plaque may begin with its detection (step 100). Several strategies are known for the detection of vulnerable plaque. As shown in FIG. 4, a detection device such as a temperature sensing device 20 may be positioned within a blood vessel lumen 22 and moved in an axial direction (e.g., shown by arrows A) thereby allowing diagnosis of a length of blood vessel 24. The device 20 may sense a blood vessel 24 temperature and determine location of a vulnerable plaque 26. The determination may be made by comparing the temperature of various portions of the blood vessel 24. The temperature of the vulnerable plaque 26 is typically one or more degrees Celsius higher than healthy vascular tissue 28 because of increased metabolic activity (i.e., inflammation). For example, a relatively normal blood vessel 24 temperature may be about 37° C. whereas the vulnerable plaque 26 may have a localized temperature as high as 40° C. As such, the temperature sensing device 20 may be used to detect the vulnerable plaque 26. Numerous devices for sensing temperature are known in the art. By way of example, device 20 may be a thermography catheter analogous to that described in U.S. Pat. No. 6,245,026 to Campbell et al. As another example, a guidewire including thermal sensors and any number of other devices known in the art may be used for sensing vessel temperature and detecting the vulnerable plaque.

In another embodiment, any number of properties specific to a vulnerable plaque may be utilized for detection. For example, vulnerable plaques generally include a localized concentration of specific lipids, proteins, and factors. Measurement of these components may facilitate detection. The detection may be achieved and/or enhanced by labeling. For example, the vulnerable plaque may be labeled with an antibody marker specific for a plaque component wherein the antibody may include a radiolabel. The radiolabel may then be detected with an appropriate detection device known in the art.

In yet another embodiment, the vulnerable plaque may be detected from external the blood vessel. For example, a device for detecting the vulnerable plaque may be positioned through an incision in the patient. The device may then detect the vulnerable plaque without the need for catheterization. During such a procedure, detection may be achieved during open surgery or in a minimally invasive manner. As another example, the vulnerable plaque may be detected external to the patient, such as with an imaging device (e.g., magnetic resonance, ultrasound, or x-ray). Those skilled in the art will recognize that the strategy for detecting the vulnerable plaque may vary from the illustrated and described methods. Numerous methods and devices for the detection of vulnerable plaque may be adapted for use with the present invention. It should be noted, however, that the detection step may be omitted during vulnerable plaque treatment. In such an instance, a treatment procedure may be performed, for example, in a region of blood vessel suspected of including vulnerable plaque(s).

Whether vulnerable plaque is detected or merely suspected, the present invention includes the step of rupturing its associated fibrous cap, for which several strategies may be used (step 101). In one embodiment, as shown in FIG. 5, a rupture device such as an incising device 30 may be positioned adjacent the vulnerable plaque 26 within the blood vessel 24 lumen 22. The device 30 may include moveable sharp edges 32 for rupturing a fibrous cap 34 of the vulnerable plaque 26. The device edges 32 may be moved in a linear or rotational manner to incise the fibrous cap 34 (i.e., in a “shaving” and/or “burring” manner; shown by arrows B and C, respectively). Numerous devices capable of incising the fibrous cap 34 are known in the art. By way of example, device 30 may be an atherectomy catheter analogous to that described in U.S. Pat. No. 4,857,045 to Rydell. Other examples of rupture devices that may be adapted for use with the present invention include the atherectomy device described in U.S. Pat. No. 5,078,723 to Dance et al. and the balloon catheter with cutting edges described in U.S. Pat. No. 5,196,024 to Barath.

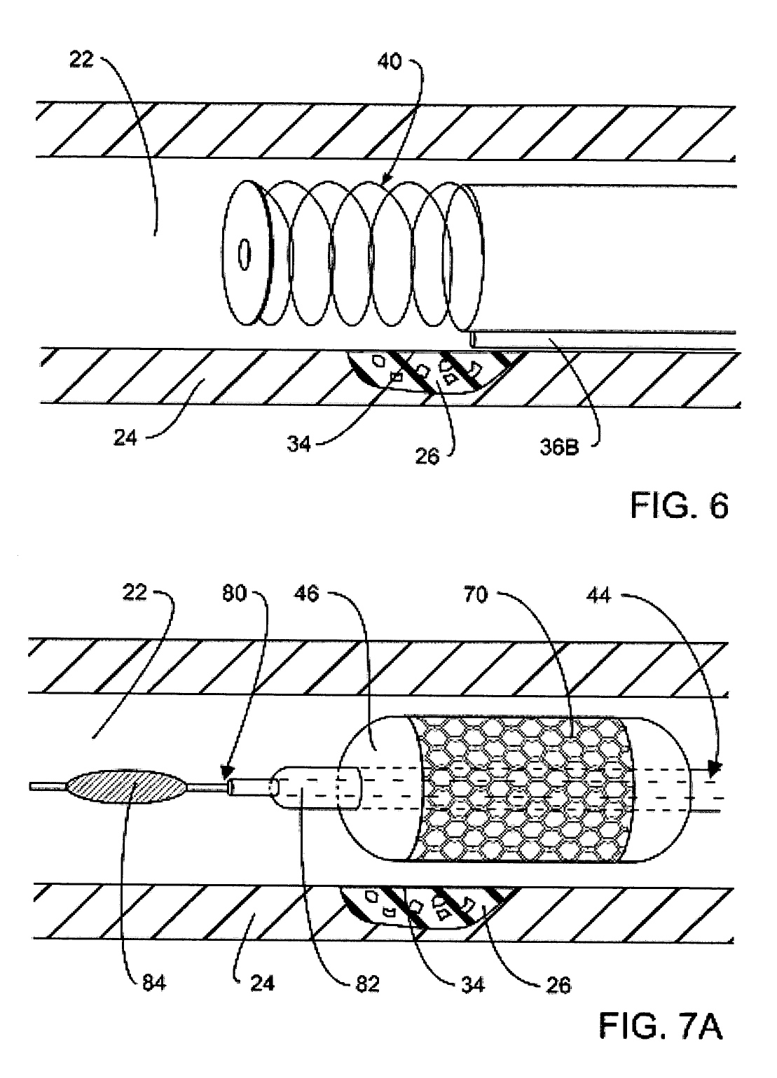

In another embodiment, as shown in FIG. 6, an ultrasonic device 40 may be positioned adjacent the vulnerable plaque 26 within the blood vessel 24 lumen 22. The device 40 may include a component for generating high frequency sound waves thereby facilitating rupture of the fibrous cap 34. Numerous devices capable of administering ultrasonic energy are known in the art. By way of example, device 40 may be a laser-driven acoustic ablation catheter analogous to that described in U.S. Pat. No. 6,203,537 to Adrian.

In yet another embodiment, a variety of devices may be used, both internal and external to the treated blood vessel, for rupturing the fibrous cap. The devices include those generating electromagnetic energy including radio wave radiation, microwave radiation, infrared radiation, visible light radiation, ultraviolet radiation, x-ray radiation, alpha radiation, beta radiation, and gamma radiation. Examples of such devices that may be adapted for use in rupturing the fibrous cap according to the present invention include a laser balloon catheter described in U.S. Pat. No. 6,224,590 to Daikuzono, a laser treatment catheter described in U.S. Pat. No. 5,916,210 to Winston, a device for in vivo radiation delivery described in U.S. Pat. No. 6,099,499 to Ciamacco, and a radio frequency atherectomy catheter described in U.S. Pat. No. 5,665,062 to Houser. These devices generally function by disrupting the physical structure of the fibrous cap thereby allowing release of the vulnerable plaque contents. Those skilled in the art will recognize that a myriad of such devices are known in the art and may be adapted for use with the present invention.

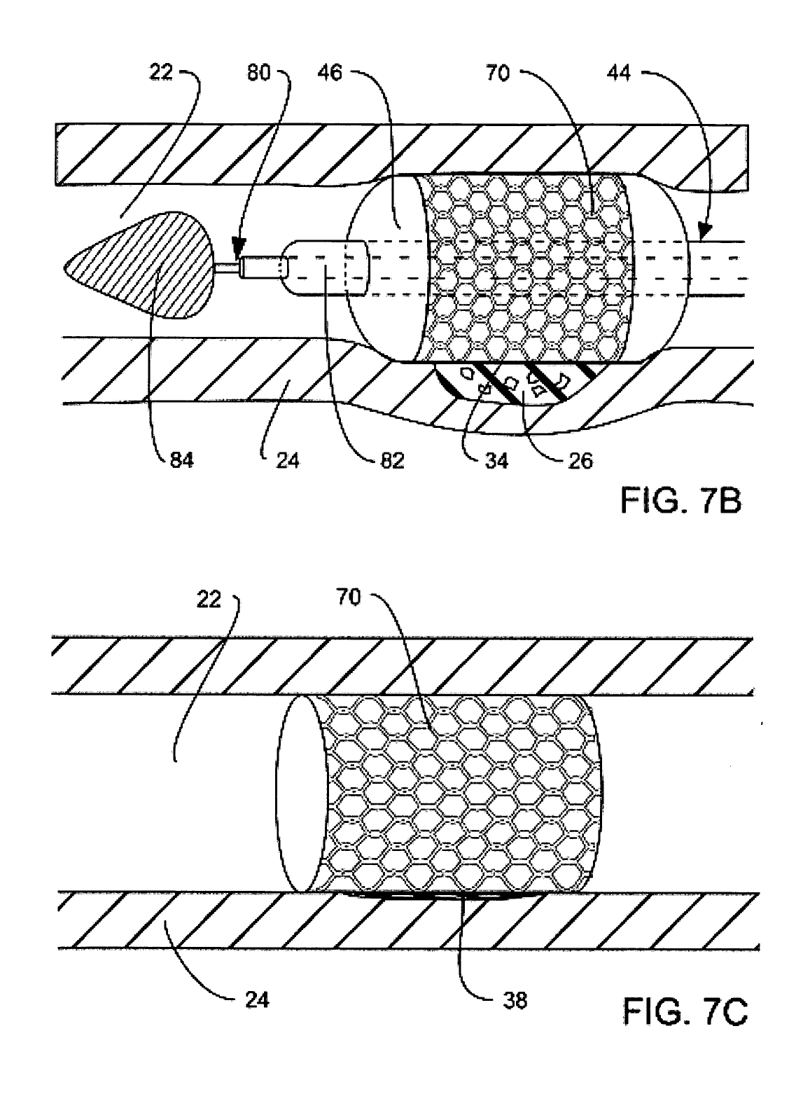

In yet another embodiment, a variety of devices may be used for generating a compressive force on the vulnerable plaque to cause rupture of the fibrous cap. In one embodiment, a balloon catheter device may be used to generate the compressive force. As shown in FIG. 7A, a rupture device such as a balloon catheter device 44 may be positioned while in a collapsed state within the blood vessel 24 lumen 22 adjacent the vulnerable plaque 26. A balloon component 46 may be inflated into contact with the vulnerable plaque 26, as shown in FIG. 7B, thereby generating a compressive force. Sufficient force exerted on the vulnerable plaque 26 may rupture the fibrous cap 34. Those skilled in the art will recognize that a myriad of devices for generating a compressive force to cause rupture are known in the art and may be adapted for use with the present invention.

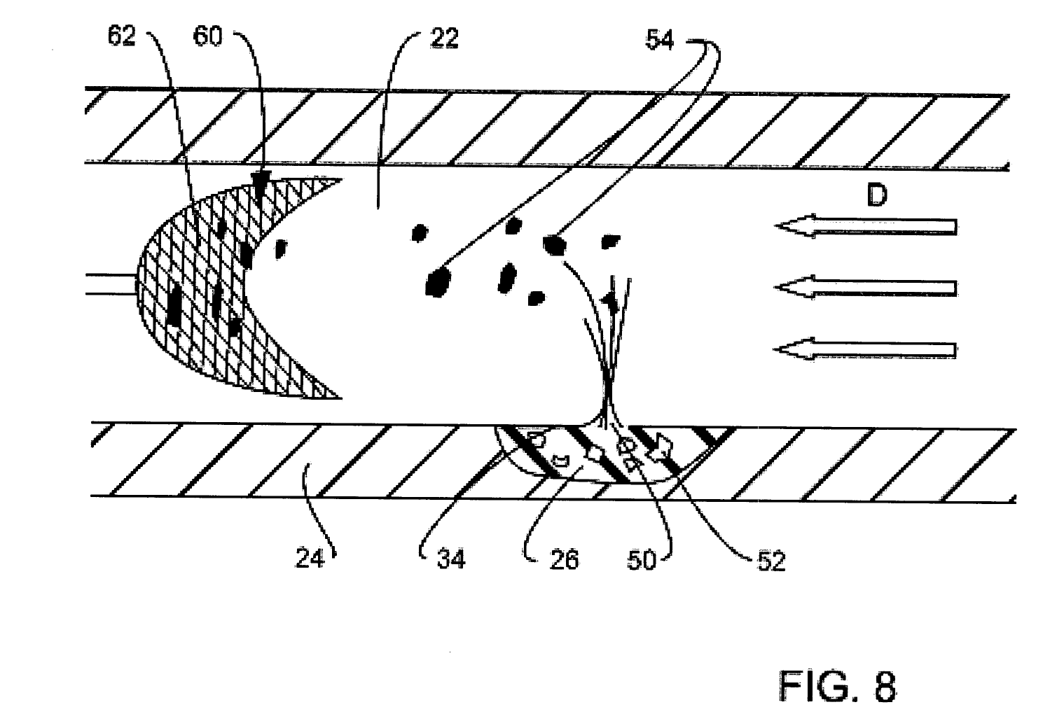

After rupture of the fibrous cap, a portion of the vulnerable plaque contents of the vulnerable plaque are allowed release into the blood vessel lumen (step 102). As shown in FIG. 8, the contents include liquid contents 50 such as lipids, growth factors, and other components that may play a role in embolus formation within the blood vessel 24. The contents may further include solid contents 52 such as hardened lipids, matrix proteins, and cells and cellular debris (e.g., macrophages, foam cells, and necrotic cells). The vulnerable plaque 26 contents may be released to the extent that the risk associated with further vulnerable plaque rupture is minimized. In other words, the contents are typically released until the plaque is stabilized and will not soon contribute to any detrimental events (e.g., heart attack or stroke).

The released contents present in the blood vessel 24 lumen 22 constitute emboli 54, or abnormal particles. The emboli 54 may further include any blood clots that may form as a result of the vulnerable plaque content release. The emboli may pose a risk to the patient provided they migrate downstream (i.e., direction shown by arrows D) the vulnerable plaque 26. Therefore, the risk may be reduced by capturing the emboli (step 103).

In one embodiment, as further shown in FIG. 8, capturing of emboli is achieved with a capture device such as a distal protection device 60 deployed downstream the vulnerable plaque 26 prior to rupture of the fibrous cap 34. The device 60 may capture the emboli 54 as they are carried by the flow of blood past the ruptured fibrous cap 34. The device 60 may include a mesh filter 62 for trapping the emboli 54 during the treatment procedure. The filter 62 may be then retracted to retain any captured emboli 54 and removed from the patient. Numerous distal protection devices for capturing emboli 54 and methods of deployment are known in the art. By way of example, device 60 may be a distal protection device analogous to that described in U.S. Pat. No. 4,873,978 to Ginsburg or U.S. Pat. No. 6,346,116 to Brooks et al.

In another embodiment, the emboli may be captured with an aspiration device. Such capture devices are typically positioned adjacent the vulnerable plaque prior to rupture of the fibrous cap. The aspiration device may provide negative pressure thereby drawing emboli through the device and may be positioned either upstream or downstream of the vulnerable plaque. Numerous aspiration devices for capturing emboli are known in the art. By way of example, the device 30 shown in FIG. 5 and device 40 shown in FIG. 6 may include an aspiration port 36a, 36b for capturing emboli. Another example of a suitable protection device includes the Export™ Catheter from Medtronic, Inc. Minneapolis, Minn. Alternatively, the aspiration device may be analogous to that described in U.S. Pat. No. 5,011,488 to Ginsburg or U.S. Pat. No. 6,398,773 to Bagaoisan et al.

After the emboli have been captured, one or more additional treatments may be performed on the ruptured vulnerable plaque (step 104). The additional treatment may include a stent expandably deployed into contact with the blood vessel. Numerous stent devices are known in the art and may be adapted for use with the present invention. The stent may be self-expanding or balloon-expandable. In one embodiment, as shown in FIGS. 7A, 7B, and 7C, a stent 70 coupled to the balloon component 46 may be deployed during the same inflation motion used to generate the compressive force on the vulnerable plaque 26. The deployed stent 70, as shown in FIG. 7C, may prevent stenosis of the blood vessel 24 adjacent a treated vulnerable plaque 38. Those skilled in the art will recognize that numerous other strategies exist for stent deployment and that the foregoing example demonstrates merely one such possibility.

The one or more additional treatments may also include cauterizing the ruptured vulnerable plaque by applying electromagnetic energy with a cauterizing device. Cauterization may reduce the possibility of vulnerable plaque re-growth and is achievable with a number of such devices known in the art.

The one or more additional treatments may also include removal of a portion of the ruptured fibrous cap. Removal of the fibrous cap, including any loose portions, may reduce the risk of it dislodging from the vulnerable plaque at a later time. In one embodiment, fibrous cap removal may be achieved with an atherectomy device.

The one or more additional treatments may also include administering at least one therapeutic agent to the patient. The therapeutic agent may be administered systemically or locally as by, for example, a catheter device positioned adjacent the vulnerable plaque. In one embodiment, as shown in FIGS. 7A and 7B, a therapeutic agent delivery device 82 including one or more lumens formed therein may be used for agent delivery. The therapeutic agent may be administered at any time during and advantageous to the treatment procedure, such as before, during, and/or after fibrous cap rupture. Examples of therapeutic agents that may be used with the treatment procedure include, but are not limited to, antiangiogenesis agents, antiarterioscierotic agents, antiarythmic agents, antibiotics, antibodies, antidiabetic agents, antiendothelin agents, antinflammatory agents, antimitogenic factors, antioxidants, antiplatelet agents, antiproliferative agents, antisense agents, calcium channel blockers, clot dissolving enzymes, growth factor inhibitors, growth factors, immunosuppressants, nitrates, nitric oxide releasing agents, vasodilators, and virus-mediated gene transfer agents.

During treatment of the vulnerable plaque, aspects of the procedure may be monitored. In one embodiment, the aforementioned devices used for various treatment steps may include a radiopaque material. The radiopaque material may be manufactured from a number of materials used for visualization in the art including platinum, gold, tungsten, metal, metal alloy, and the like. The radiopaque material may be visualized by fluoroscopy, IVUS, and other methods known in the art for monitoring the positioning, deployment, and function of the treatment devices.

In another embodiment, capture of the emboli may be monitored. For example, captured emboli may be visually monitored as they pass through an aspiration device. The absence of emboli passing through the device may signify to the treating physician that the treatment procedure may continue.

The treatment procedure may conclude once the vulnerable plaque has been satisfactorily treated according to the described methodology. Additional treatments may be administered on other vulnerable plaque(s) during the same procedure and/or on a previously treated plaque during a subsequent procedure. Upon completion of the treatment, the patient may remain under observation in the clinical setting. As such, any potential complications may be addressed immediately thereby reducing risk to patient.

The functions ascribed to the aforementioned devices and system may be achieved with a single or with multiple devices. In one embodiment, as shown in FIGS. 7A and 7B, a combination device 80 may be used to implement the system and method of the present invention. The combination device 80 combines balloon catheter 44 including balloon component 46 for compressing the vulnerable plaque 26, stent 70, therapeutic agent delivery device 82 for delivering therapeutic agent(s), and an expandable/collapsible distal protection device 84. The combination device 80 may perform multiple treatment steps thereby increasing the efficiency of, and potentially reducing the length of, the procedure. The combination device 80 illustrates merely one example of a myriad of possible devices for implementing the method and system of the present invention. Those skilled in the art will recognize that the method and system for treating vulnerable plaque may include and/or exclude numerous device components and that the nature of those components may vary as well.

While the embodiments of the invention disclosed herein are presently considered to be preferred, various changes and modifications may be made without departing from the spirit and scope of the invention. The system, device(s), and method of utilizing the same are not limited to any particular design or sequence. Specifically, the system and device components, procedure step order, and method of achieving the same may vary without limiting the utility of the invention. Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. A method of treating a vulnerable plaque associated with a blood vessel of a patient, the method comprising:
rupturing a fibrous cap of the vulnerable plaque;
releasing a portion of liquid contents of the vulnerable plaque into a blood vessel lumen as a result of the fibrous cap rupture; and
capturing at least one of any emboli present within the blood vessel as a result of the fibrous cap rupture.

2. The method of claim 1 wherein rupturing the fibrous cap comprises compressing the vulnerable plaque.

3. The method of claim 1 wherein rupturing the fibrous cap comprises incising the fibrous cap.

4. The method of claim 1 wherein rupturing the fibrous cap comprises administering ultrasonic energy.

5. The method of claim 1 wherein rupturing the fibrous cap comprises administering electromagnetic radiation energy.

6. The method of claim 5 wherein the electromagnetic radiation is selected from a group consisting of radio wave radiation, microwave radiation, infrared radiation, visible light radiation, ultraviolet radiation, x-ray radiation, alpha radiation, beta radiation, and gamma radiation.

7. The method of claim 1 wherein the fibrous cap is ruptured with a device selected from the group consisting of a balloon device, a lasing device, a heating device, an ultrasonic device, a radio frequency device, a device for delivering radiation, and an incising device.

8. The method of claim 1 wherein capturing the emboli comprises deploying a distal protection device.

9. The method of claim 1 wherein capturing the emboli comprises aspirating the emboli.

10. The method of claim 1 further comprising detecting the vulnerable plaque.

11. The method of claim 10 wherein detecting the vulnerable plaque comprises labeling the vulnerable plaque.

12. The method of claim 10 wherein detecting the vulnerable plaque comprises determining a temperature of the vulnerable plaque.

13. The method of claim 1 further comprising releasing a portion of solid contents of the vulnerable plaque into the blood vessel lumen as the result of the fibrous cap rupture.

14. The method of claim 1 further comprising stenting the blood vessel adjacent the vulnerable plaque.

15. The method of claim 1 further comprising cauterizing the vulnerable plaque.

16. The method of claim 1 further comprising removing a portion of the ruptured fibrous cap.

17. The method of claim 1 further comprising monitoring the treatment of the vulnerable plaque.

18. The method of claim 1 further comprising administering at least one therapeutic agent to the patient.

19. The method of claim 18 wherein the therapeutic agent is selected from a group consisting of antiangiogenesis agents, antiarteriosclerotic agents, antiarythmic agents, antibiotics, antibodies, antidiabetic agents, antiendothelin agents, antinflammatory agents, antimitogenic factors, antioxidants, antiplatelet agents, antiproliferative agents, antisense agents, calcium channel blockers, clot dissolving enzymes, growth factor inhibitors, growth factors, immunosuppressants, nitrates, nitric oxide releasing agents, vasodilators, and virus-mediated gene transfer agents.

20. A system for treating a vulnerable plaque associated with a blood vessel of a patient, the system comprising:
a rupture device that ruptures a fibrous cap of the vulnerable plaque; and
a capture device that captures at least one embolus within the blood vessel.

21. The system of claim 20 wherein the rupture device is selected from a group consisting of a balloon device, a lasing device, a heating device, an ultrasonic device, a radio frequency device, a device for delivering radiation, and an incising device.

22. The system of claim 20 wherein the capture device is selected from a group consisting of a distal protection device and an aspiration device.

23. The system of claim 20 further comprising a detection device that detects the vulnerable plaque.

24. The system of claim 23 wherein the detection device comprises a thermal sensor.

25. The system of claim 20 further comprising a stent operably coupled to the rupture device.

26. The system of claim 20 further comprising a cauterizing device that cauterizes the vulnerable plaque.

27. The system of claim 20 further comprising a therapeutic agent delivery device.
